(19) United States
(12) Patent Application Publication
(10) Pub. No.: US 2022/0032023 A1
(43) Pub. Date: Feb. 3, 2022

(54) ULTRA-SHARP MICRONEEDLE

(71) Applicant: THE TRUSTEES OF COLUMBIA UNIVERSITY IN THE CITY OF NEW YORK, New York, NY (US)

(72) Inventors: Aykut AKSIT, New York, NY (US); Jeffrey W. KYSAR, New York, NY (US); Anil K. LALWANI, New York, NY (US)

(21) Appl. No.: 17/503,304

(22) Filed: Oct. 16, 2021

Related U.S. Application Data
(63) Continuation-in-part of application No. PCT/US20/28497, filed on Apr. 16, 2020.
(60) Provisional application No. 63/152,032, filed on Feb. 22, 2021.
(60) Provisional application No. 63/151,983, filed on Feb. 22, 2021.
(60) Provisional application No. 62/965,701, filed on Jan. 24, 2020.
(60) Provisional application No. 62/834,807, filed on Apr. 16, 2019.

(51) Int. Cl. A61M 31/00; A61M 1/00

(52) U.S. Cl. CPC A61M 31/00; A61B 10/0045; A61M 2210/0662; A61M 1/87

(57) ABSTRACT

A microneedle having a body including a base, shaft and pointed tip, and at least one lumen disposed within the shaft. The lumen having an opening that is offset from the tip and open to the exterior of the microneedle.

### RELATED APPLICATIONS

This application is a continuation-in-part of International Application PCT/US2020/028497 filed Apr. 16, 2020, which claims benefit of priority to U.S. Provisional Application Ser. No. 62/834,807 filed Apr. 16, 2019 and 62/965,701 filed Jan. 24, 2020. This application also claims benefit of priority to U.S. Provisional Application Ser. No. 63/152,032 filed Feb. 22, 2021 and 63/151,983 filed Feb. 22, 2021. The content of each of the above-identified applications is incorporated herein by reference thereto.

### GOVERNMENT FUNDING

This invention was made with government support under contract no. R01-DC014547-03 and grant number 753600XOTO Otolaryngology GG011895 01 70003 AKL2144, awarded by the National Institutes of Health. The government may have certain rights in the invention.

### FIELD

The disclosed subject matter describes an apparatus for controlled precision of perforating a thin membrane and delivery or aspiration of fluid. More particularly, the subject matter described is an ultra-sharp microneedle configured to produce a controlled and precisely shaped and sized perforation in a thin membrane, for example, of the inner ear. Further, the needle apparatus may be configured for safe, controlled, local injection delivery to or aspiration of fluid across the thin membrane.

### BACKGROUND

Symptoms such as hearing loss, tinnitus, and vertigo—which can have debilitating effects on one's quality of life—are quite prevalent in the general population. These symptoms, either alone or in combination, can be reflective of underlying otologic disorders that necessitate specific medical or surgical intervention. Despite previous research and innovation, effective treatments for common inner ear illnesses such as sudden or progressive sensorineural hearing loss (SNHL) and Meniere's disease have remained particularly elusive, in part due to the anatomic inaccessibility of the cochlea. Protected by one of the hardest bones in body, the cochlea is nearly an impenetrable structure frustrating both bacteria and clinician trying to gain access to it. As a result, a means for reliable delivery of agents into the inner ear for therapeutic purposes remains a formidable challenge.

Various methods of intracochlear delivery have been developed to access this elusive anatomical space, but these methods all involve breaching the inner ear and consequently risk hearing impairment from traumatic disruption of the cochlea. Thus, despite extensive research and promising new technologies, a safe, reliable method for intracochlear delivery remains to be developed. Likewise, the challenge of delivering large quantities of medication or sampling of large volumes of biological fluids remains.

Due to pharmaceutical limitations, such as solubility and dosage, it is of great interest to displace large amounts of liquids in the cochlea. Since the cochlea is small in scale, it becomes difficult to inject “large” amounts since an amount equal to what has been introduced needs to flow out. If the outflow happens slowly, this can increase intracochlear pressure, possibly causing harm to the delicate structures of the inner ear. Hence, it is important that this process is facilitated in a controlled way. For example, it would be useful to be able to inject a liquid therapeutic agent into the inner ear while simultaneously withdrawing an equal amount of liquid from the inner ear.

In order to acquire samples for diagnostic purposes, volumes of greater than 1 μL are typically necessary for analysis. Volumes of this size are quite significant with regards to the total size of the cochlea, and can create problems if not replaced, especially with volumes over 3 μL. This can be crucial for some potential diagnostic tests which require large amounts of biofluids to be processed. Artificial perilymph is known to be a good substitute for perilymph, and can be created easily. It would be useful to be able to withdraw a sample of perilymph from a subject's inner ear while simultaneously replacing it with an equal volume of artificial perilymph.

### SUMMARY

An ultra-sharp microneedle is provided. The microneedle is sized for use in the eye, ear and central nervous system. In some applications, the microneedle has multi-lumen for simultaneous delivery and sampling into a fixed volume of biological tissue.

The microneedle generally includes a base disposed at a proximal end of the microneedle body and a tip disposed at the distal end of the microneedle body. A cylindrical shaft having a longitudinal body is disposed between the base and the tip. In some embodiments, the base, shaft and tip may be integral. The base can have a cylindrical shape configured to mount onto a medical instrument, such as a syringe. For example, the microneedle may be mounted on a 30 gauge syringe. The base has a width greater than the width of the tip, and in some embodiments is also greater than the shaft. In other words, for a cylindrical base and shaft, the diameter of the base is greater than the diameter of the shaft. Further the base is sized to mount and securely engage an instrument such as a driver or syringe. In this regard, mounting on a hollow or solid material of any cross-sectional shape may be achieved. Likewise, the mount may also consist of one or more lumen that would allow the delivery or aspiration of varying amounts of material through each lumen.

The at least one lumen defines a channel that provides fluid communication and passage through at least the shaft of the needle. The lumen is disposed within at least the shaft cylindrical body of the microneedle. An opening is defined at the distal end of the lumen body and also at a loci of the shaft body to communicate to the exterior of the microneedle. The opening is offset from a central longitudinal axis of the tip. The central longitudinal axis of the tip may also be the central longitudinal axis of the shaft. In this regard, the opening would also be offset from the shaft central axis.

The at least one lumen has a body may extend through the base to the shaft of the microneedle. Thus, the lumen may terminate prior to or proximate the tip of the microneedle.

The tip includes a sharp, pointed apex. The tip may have 500-nm curvature of radius. The shaft may have a total length of about 350 microns. The maximum tip diameter may be about 10-20 microns. In some embodiments, there is one sole lumen, in other embodiments, there are at least a first and second lumen.

The shaft in some embodiments has a tapered longitudinal, cylindrical body configured with a taper along a portion of its length.

In a second embodiment, the a microneedle comprising a base, a shaft, a sharp tip and two or more lumens wherein a first lumen provides fluid communication and passage from a first portion of the base of the microneedle to a first locus proximate to the tip of the microneedle and a second lumen provides fluid communication and passage from a second portion of the base of the microneedle to a second locus on the shaft of the microneedle. The first locus and the second locus are diametrically opposed on the microneedle, e.g., shaft. The microneedle may have a maximum tip diameter of about 10-20 microns.

In some embodiments, the microneedle shaft is configured with a taper along a portion of its length. The taper comprises a gradual taper having a gradual decrease in diameter along the length of the microneedle. The taper may comprise a stepped taper with abrupt changes in diameter that serve as reinforcing ribs or ledges.

The microneedle distal portion comprises a narrow sharp tip, the proximal end may comprise a wide base and a shaft between the tip and the base. The base of the distal portion may comprise one or more projections or barbs that engage the distal side of the membrane after penetration through the membrane and is held in place thereby. The base may be configured to physically engage a driver device capable of creating temporary perforations in an anatomic membrane. The may be configured to penetrate an anatomic membrane and simultaneously inject a first liquid into the distal side of the membrane and withdraw a second liquid from the distal side of the membrane. The anatomic membrane may be a membrane of the inner ear.

In some embodiments, one lumen is an aspirating lumen which is connected to a suction device, such as a vacuum source, to suction a fluid from the distal side of the membrane. The same or a second lumen may an injection lumen which is connected to an injection device, such as a syringe, to inject a fluid into the distal side of the membrane.

In another aspect, a medical device comprises the microneedle described in any of the embodiments above, wherein the base the microneedle is physically engaged with a driver device capable of creating temporary perforations in an anatomic membrane and simultaneously inject a first liquid into the distal side of the membrane and withdraw a second liquid from the distal side of the membrane. The membrane may be the round window membrane of an inner ear.

The microneedle and the driver comprise separate components that are engaged to each other to define a modular system. The medical device may further comprise an indicator to indicate when the membrane is fully penetrated by the microneedle.

In another aspect, a method of delivering a therapeutic agent through an anatomic membrane is provided. The method includes positioning a microneedle proximate the membrane wherein the microneedle is configured to penetrate the membrane. Perforating the anterior side of the membrane, dispensing a therapeutic agent at the perforation from a first lumen and withdrawing an amount of fluid from the posterior side of the membrane, wherein the amount of fluid withdrawn is substantially equivalent to the amount of the therapeutic agent injected. The withdrawal and aspiration steps can occur simultaneously.

In some embodiments, the method delivers a therapeutic agent into the cochlea and comprises positioning at least one microneedle proximate the round window membrane. The microneedle is configured to penetrate the round window membrane, and the method includes perforating the round window membrane, dispensing a therapeutic agent at said perforation(s), and withdrawing perilymph from the inner ear.

In some embodiments, the microneedle can sample microliter amounts of perilymph fluid, such as aspirating 1 microliter of perilymph fluid. Advantageously, the sampling does not cause lasting damage to hearing and heals within hours, such as 72 hours after perforation. Perforations with the microneedle described herein may also result in no hearing loss.

In another aspect, a method of sampling a bodily fluid through an anatomic membrane. The method comprises positioning a microneedle proximate the membrane wherein the microneedle is configured to penetrate the membrane, perforating the membrane, sampling the bodily fluid at said perforation via a first lumen by withdrawing an amount of fluid from the posterior side of the membrane, and dispensing a second fluid into the posterior side of the membrane that is substantially equivalent or equivalent to the amount of bodily fluid withdrawn via a second lumen.

In one embodiment, the method is for sampling a subject's perilymph from the cochlea and comprises positioning at least one microneedle proximate the round window membrane wherein the microneedle is configured to penetrate the round window membrane; perforating the round window membrane; withdrawing perilymph from the inner ear via a first lumen; and dispensing artificial perilymph into the distal side of the membrane that is substantially equivalent or equivalent to the amount of the subject's perilymph withdrawn via a second lumen.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic illustration of a microneedle in accordance with the disclosed subject matter.

FIG. 2 is another schematic illustration of the microneedle of FIG. 1.

FIG. 3 is another schematic illustration of an offset lumen within the shaft of the microneedle of FIG. 1

FIG. 4 shows perspective views of another embodiment of a microneedle in accordance with the disclosed subject matter.

FIGS. 5A-5D show schematic views of a microneedle in accordance with the disclosed subject matter.

FIGS. 6A and 6B show top and cross-section schematic views of a microneedle according to an embodiment of this disclosure.

FIGS. 7A-7D show schematic views of a mold for fabricating a microneedle by two-photon templated electrodeposition according to an embodiment of this disclosure.

FIG. 8 shows top and cross-section schematic views of a microneedle mold according to an embodiment of this disclosure, similar to the embodiment shown in FIGS. 7A-7D.

FIG. 9 shows a view of an array of microneedles on a unitary base according to an embodiment of this disclosure.

FIG. 10 illustrates a five-needle array of microneedles according to an embodiment of this disclosure.

FIG. 11 illustrates a five-needle array approaching a guinea pig RWM.

FIG. 12 illustrates the microneedle array perforation area for the 3-, 4-, and 5-microneedle arrays according to an embodiment of this disclosure.

FIG. 13 illustrates the force (mN) vs. displacement (microns) of the 5 needle array according to an embodiment of this disclosure.

FIGS. 14-15 illustrate the confocal images of the RWM showing perforations made by the 4-needle array according to an embodiment of this disclosure.

FIG. 16A illustrates a longitudinal cross section of a microneedle according to an embodiment of this disclosure.

FIG. 16B illustrates an axial cross section of the microneedle illustrated in FIG. 16A, taken along lines 16B-16B of FIG. 16A, according to an embodiment of this disclosure.

FIG. 16C illustrates an axial cross section of the microneedle illustrated in FIG. 16A, taken along lines 16C-16C of FIG. 16A, according to an embodiment of this disclosure.

FIGS. 17-18 illustrate the composition of guinea pig perilymph protein by function categories.

FIG. 19 illustrates Mean CAP threshold shifts at 72 h compared to baseline, for all tested frequencies.

FIGS. 20-21 illustrate DPOAE at the frequency 2f1-f2 in response to a 70 dB stimulus averaged over all survival experiments at 0 h after perforation.

### DETAILED DESCRIPTION

Reference has been made in detail to select embodiments of the disclosed subject matter, examples of which are illustrated in the accompanying drawings. The method and corresponding steps of the disclosed subject matter will be described in conjunction with the detailed description of the system.

In one aspect, a microneedle for controlled precision of perforating a thin membrane and delivery or aspiration of fluid is provided. The microneedle may be configured to produce a controlled and precisely shaped and sized perforation in a thin membrane, for example, of the inner ear. The microneedle may achieve a safe, reliable method for intracochlear delivery.

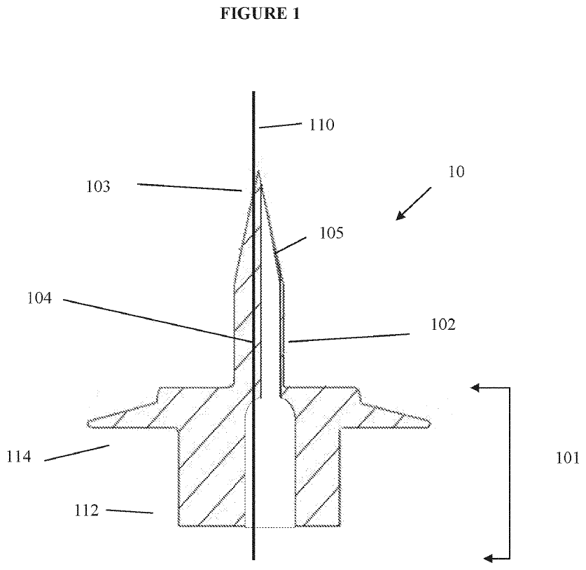

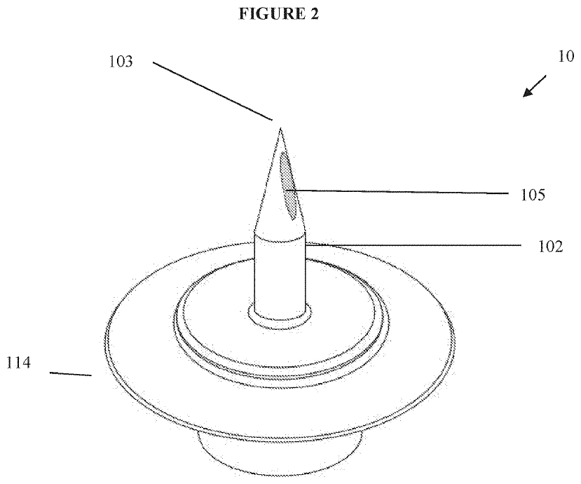

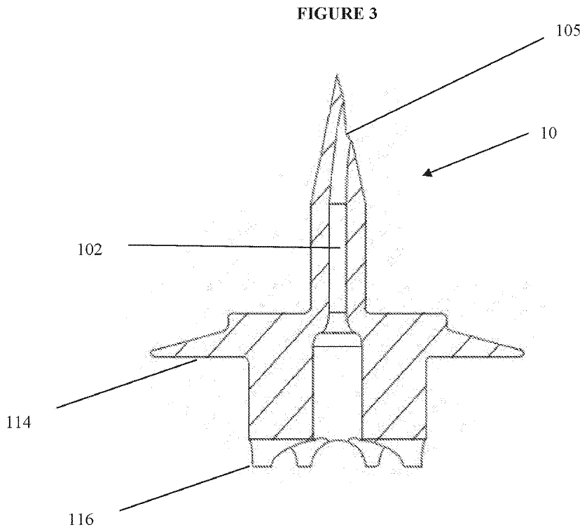

Referring to FIG. 1, in one embodiment the microneedle 10 generally comprises a base 101, shaft 102 and ultra-sharp, pointed tip 103. A lumen 104 having a longitudinal body is disposed within the shaft 102. The lumen 104 defines a channel for fluid communication and passageway from a first portion of the microneedle to a first opening 105 defined at the distal end of the lumen and a locus of the shaft 102, said opening 105 proximate to the end of tip 103. In this regard, first opening 105 is offset from a center longitudinal axis 110 of the shaft 102 and tip 103. Referring to FIGS. 2 and 3, in some embodiments, first opening 105 may have a diameter of about 30 microns, and first opening 105 is offset from the center longitudinal axis 110 of the shaft and/or tip apex by about 15-30 microns, e.g. 25 microns.

Referring to FIG. 1, the base includes a first section defined by a cylindrical body 112 and skirt 114. Skirt 114 may function as a stop in certain applications. In some embodiments, base 101 further includes leg members 116, as shown in FIG. 3.

Referring to FIG. 3, the shaft 102 has a length extending from the base 101 to the tip 103. The first section is defined by a cylindrical, hollow body having a substantially uniform diameter. The second section is defined by a conical, shaped body defined by a taper along a portion of its length terminating at a sharp or pointed tip. In some embodiments, the tip is closed, i.e., does not have a port or opening. In some embodiments depending on the application, the microneedle may a maximum tip diameter of about 10-20 microns. Lumen 104 extends from shaft 102 through to or through at least a portion of base 101, and has a second opening (not shown) disposed in base 101.

Base 101 is configured to mount to a commercially available standard blunt syringe. In this regard, second opening of lumen is in fluid communication with syringe and provides a channel for passage of therapeutic agent from syringe through lumen 102 to opening 105 and exterior of microneedle 10. Introduction of the lumen 104 through substantially the entire the microneedle body enables direct injection of fluids, such as therapeutics agents, from inside the syringe, and/or allows sampling of fluids from body, such as the inner ear, into the syringe structure.

In some embodiments, the outer diameter of shaft 102 is 100 microns and the inner diameter is 35 microns, the height of the microneedle from proximal end of base 101 to distal end of tip 103, or alternatively, from proximal end of shaft 102 to distal end of tip 103 is not greater than 350 microns. Thus, the microneedle may be configured to penetrate a thin membrane with tip 103 and is suitably sized to fit within small spaces of the anatomy, such as the cochlear, or other sites, for local delivery of therapeutic agent or aspirate fluid, such as perilymph fluid from the inner ear. In some embodiments, the ultra-sharp microneedle has a 500-nm tip radius of curvature.

In another aspect, a multi-lumen microneedle is provided. The multi-lumen structure enables, for example, a sample of perilymph from a subject's inner ear to be withdrawn, while simultaneously replacing it with an equal volume of artificial perilymph. Thus, this application, which should not be construed to limit the microneedle to this application, permits acquisition of samples from the cochlea for diagnostic purposes, for example in volumes of greater than 1 μL, for example 3 μL or more, while simultaneously replacing the withdrawn biofluid with an equal or substantially equal volume of fluid, such as artificial biofluid. Thus, increased intracochlear pressure, and harm to the delicate structures of the inner ear may be minimized, inhibited or prevented.

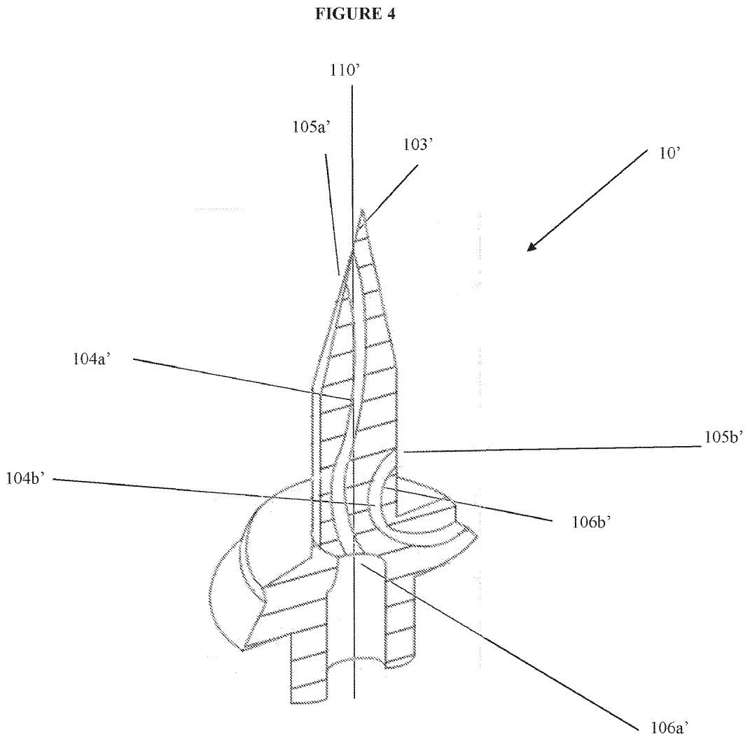

Referring to FIG. 4, in another aspect, a multi-lumen microneedle 10′ having a first lumen 104a′ and a second lumen 104b′ disposed within shaft 102′ of the microneedle is provided. The first lumen 104a′ has a first opening 105a′ and second opening 106a′ and defines a first channel therebetween. The first channel defined by first lumen provides fluid communication and passageway from a first portion of the microneedle to first opening 105a′ defined at the distal end of the lumen 105a′ and at a first locus of shaft 102′ proximate the tip 103′. As shown the shaft may be hollow as depicted in FIG. 1-3 or only partially hollow, for example, hollow portions created by the one or more lumen, as depicted in FIG. 4. The one or more lumen may extend through the shaft or the entire microneedle including base 101. Each individual lumen could run to near the tip of the microneedle, or it could run only a portion of the way up the shaft as described.

Opening 105a′ is offset from the center longitudinal axis 110′ of the shaft 102′ and tip 103′. The second lumen 104b′ defines a second channel for fluid communication and passageway from a second portion of the microneedle to opening 105b′ defined at the distal end of the lumen 104b′ and at a second locus of shaft 102′ proximate tip 103. In one embodiment, first opening 105a′ associated with lumen 104a′ is disposed at a first locus that is diametrically opposed to the disposition of the second opening 105b′ of second lumen 104b′ at the second locus. Lumen 104b′ further includes opening 106b′ extending through base 101′, for example, through the skirt 114′ to the exterior of the microneedle 10′. In this regard, lumen 104b′ may withdraw fluid from a subject after penetration with microneedle 10′ and dispel said withdrawn fluid to the exterior of the subject and microneedle 10′. Although FIG. 4 illustrates a dual-lumen needle it is understood that the needle can include three or more lumens.

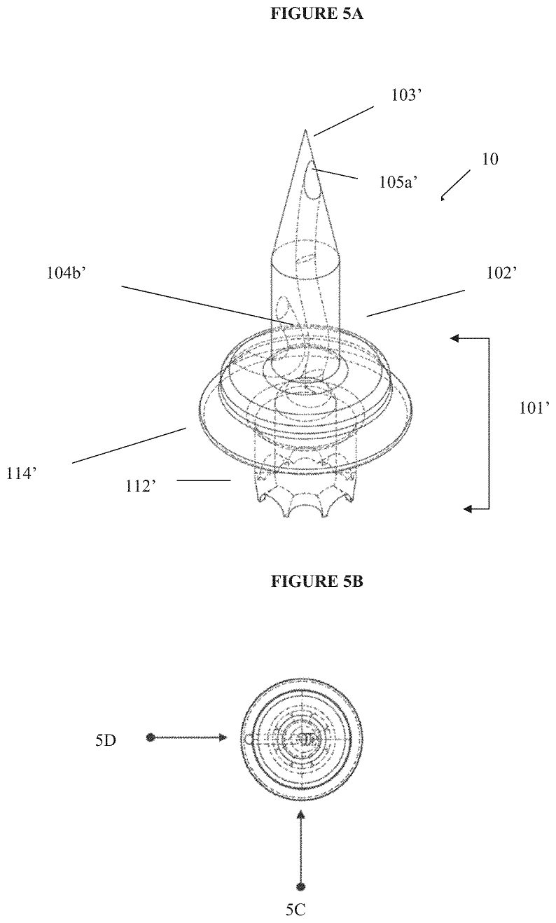

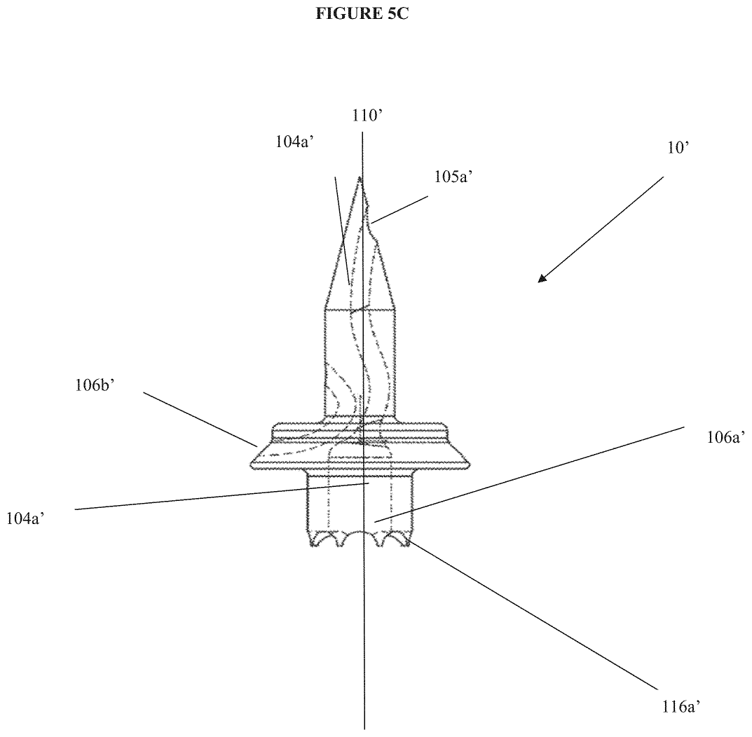

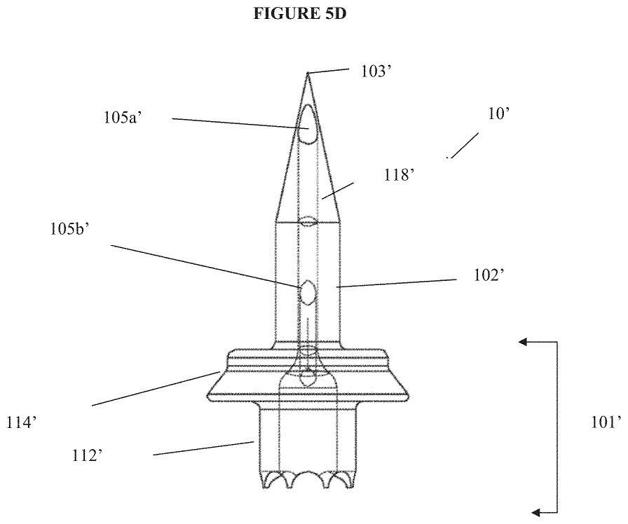

FIG. 5A-5D show schematic views of a microneedle according to an embodiment of this disclosure, similar to the embodiment shown in FIG. 4, wherein solid lines denote visible aspects of the microneedle and dashed lines denote invisible aspects of the microneedle, for example portions of the microneedle on the interior of the microneedle. Referring to FIG. 5A a first lumen 104a′ provides fluid communication and passage from a first portion of the base of the microneedle to a first locus proximate to the tip of the microneedle and a second lumen provides fluid communication from a second portion of the base of the microneedle to a second locus on the shaft of the microneedle. FIG. 2B shows a top schematic view of the microneedle. FIG. 5C shows a side schematic view of the microneedle, viewed along line 5C shown in FIG. 5B. FIG. 5D shows a side schematic view of the microneedle, viewed along line 5D shown in FIG. 5B.

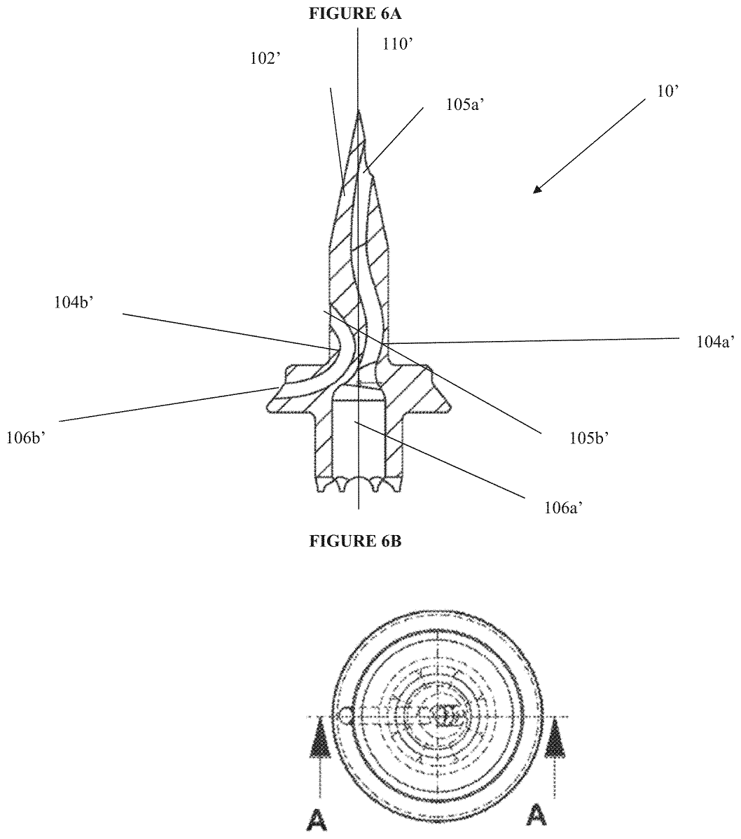

FIG. 6 shows top and cross-section schematic views of a microneedle according to an embodiment of this disclosure, similar to the embodiment shown in FIG. 1. The upper image shows a top schematic view of the microneedle, wherein solid lines denote visible aspects of the microneedle and dashed lines denote invisible aspects of the microneedle, for example portions of the microneedle on the interior of the microneedle. The bottom image shows a cross-section view of the microneedle wherein the structure of the microneedle has been cut away along line A-A shown in the upper image. Solid (i.e. polymerized) portions of the microneedle are shown as hashed areas. The lumens are shown as non-hashed.

Microneedles according to embodiments of this disclosure may have the following dimensions as indicated in Table 1:

TABLE 1

| Microneedle portion | Range | Example |
|---|---|---|
| Tip radius of curvature | less than 3 μm | 1.5 μm |
| Shaft diameter | 75 to 150 μm | 100 μm |
| Base diameter | 300 to 450 μm | 405 μm |
| Needle height | 300 to 500 μm | 430 μm |
| Total structural height | 400 to 650 μm | 585 μm |

In another aspect, a method of delivering a therapeutic agent through an anatomic membrane is provided. The method comprises positioning the microneedle, described herein, proximate the membrane wherein the microneedle is configured to penetrate the membrane; perforating the membrane; dispensing a therapeutic agent at said perforation from a first lumen; and withdrawing an amount of fluid from the distal side of the membrane that is equivalent to the amount of the therapeutic agent injected. For example, the method may deliver a therapeutic agent into the cochlea and comprises positioning at least one microneedle proximate the round window membrane wherein the microneedle is configured to penetrate the round window membrane; perforating the round window membrane; dispensing a therapeutic agent at said perforation(s); and withdrawing perilymph from the inner ear.

In another aspect, thus, a method of sampling a bodily fluid through an anatomic membrane. The method comprises positioning a microneedle proximate the membrane wherein the microneedle is configured to penetrate the membrane; perforating the membrane; sampling the bodily fluid at said perforation via a first lumen by withdrawing an amount of fluid from the distal side of the membrane; and dispensing a second fluid into the distal side of the membrane that is equivalent to the amount of bodily fluid withdrawn via a second lumen.

In one embodiment, the method is for sampling a subject's perilymph from the cochlea and comprises positioning at least one microneedle proximate the round window membrane wherein the microneedle is configured to penetrate the round window membrane; perforating the round window membrane; withdrawing perilymph from the inner ear via a first lumen; and dispensing artificial perilymph into the distal side of the membrane that is equivalent to the amount of the subject's perilymph withdrawn via a second lumen.

Since the cochlea has a fixed volume, fluid could be aspirated from the lumen exiting near the base of the microneedle at the same time a therapeutic is injected out of the lumen exiting near the tip. Alternatively if the goal is aspiration without injection of a therapeutic, aspiration could occur from one of the lumina while artificial perilymph (or another “inert” substance) is injected through the other. This method could maintain the same volume of fluid within the cochlea thus minimizing pressure changes within the cochlea during such interventions. Or, the aspiration and injection flow rates and tidal volumes could be prescribed not to be the same to introduce purposely a change in pressure in the closed volume. Of course these same principles would apply if more than two lumina are in the microneedle.

The microneedles decribed herein may be fabricated using Two Photon Polymerization Lithography (2PP) techniques. (Nanoscribe, GmbH) (Material used: IP-S photoresist by Nanoscribe GmbH). Two-Photon Polymerization (2PP) is a new technology suited for the fabrication of nearly arbitrary 3D micro- and macro-structures. Many items can be produced by either subtracting materials, such as machining or carving, or by adding materials, for example 3D printing. With two-photon polymerization, a photosensitive material is provided and very short pulses of laser light are used to build structures within that material. Once the structure is done, the non-polymerized material is removed, by for example washing the workpiece with a suitable solvent. Using this method, complex shapes can be created that would be impossible with routine 3D printing.

In contrast to conventional 3D printing, 2PP is not limited to a layer-wise fabrication of the desired structure. In fact, it is an inherently 3D process technology. 2PP technology is based on a process where light triggers a chemical reaction, leading to polymerization of a photosensitive material. Polymerization is a process in which monomers or weakly cross-linked polymers (liquid or solid) interconnect and form three-dimensional network of highly cross-linked polymer (solid). Photoinitiators, molecules which have low photo dissociation energy, are often added in order to increase the material photosensitivity. Absorption of a photon in the photoreactive material leads to a bond cleavage (photo dissociation) and formation of highly reactive radicals. Absorption of a UV photon breaks C—C bond and results in the formation of two radicals, which react with the monomer, e.g. methyl methacrylate, and initiate radical polymerization. The reaction is terminated when two radicals react with each other.

2PP is made possible by tightly focusing femto-second laser pulses into a transparent photopolymer. The femtosecond laser radiation induces a highly localized chemical reaction leading to polymerization of the photosensitive material. The photon-polymerization triggered by the laser pulses is strongly confined to the focal volume as the underlying process. Two-Photon Absorption (TPA) can only occur if the intensity is sufficiently large. As a consequence, the site of the reaction i.e. the solidification of the material is located in the tiny focal volume.

For 3D fabrication, the focal volume is scanned in 3D space followed by a solvent wash to separate the rest of the (nonpolymerized) photopolymer from the solidified 3D structure. In addition to being a true 3D process, 2PP is not limited to the optical diffraction limit as the photon-polymerization is a threshold process. By adjusting the photon dose only slightly beyond the polymerization threshold, feature sizes in the order of 100 nm can be achieved.

Freeform microlenses and microoptics used in 2PP allow for the fabrication of more complex topographies, particularly non-spherical or free-form designs. The photosensitive material is typically supported on a substrate such as silicon, borosilicate glass or fused silica, and the substrate may be transparent or opaque. Following creation of the microneedle(s) by 2PP and removing the nonpolymerized material, the microneedle(s) can be separated from the substrate. In some embodiments, the microneedle can be separated from the substrate by leaving a portion of the photopolymer adjacent to the substrate nonpolymerized, which is then removed by the solvent wash. Alternatively, portions of the microneedle, such as a portion of the base, may comprise a substrate that the polymerized microneedle remains adhered to after fabrication. Because the fabrication process may take a significant amount of time, such as hours or even days, it is desirable to perform the 2PP process in a way that can make more than one microneedle at a time.

It may also be possible to create similar microneedles using two-photon templated electrodeposition, which means the microneedles will then be fully metallic. This fabrication process directly links three-dimensional (3D) modeling and simulation with microscale printing and replication. The process involves micromolds fabricated by 3D stereolithography directly from CAD drawings, which are then used to provide shaped metal microneedles using electrodeposition techniques. Alternatively, the mods may be prepared by 2PP.

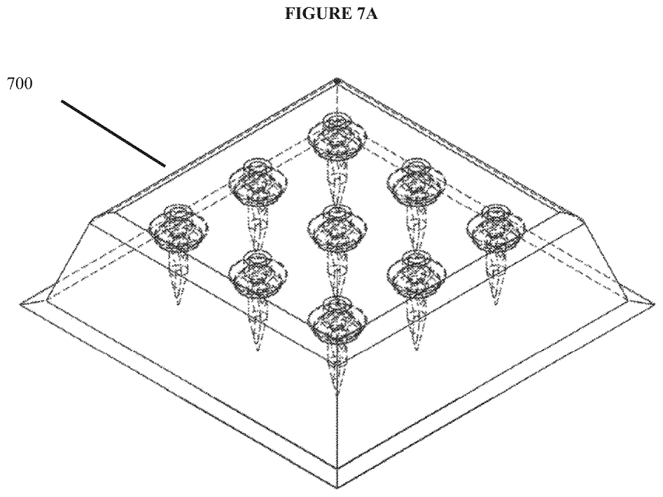

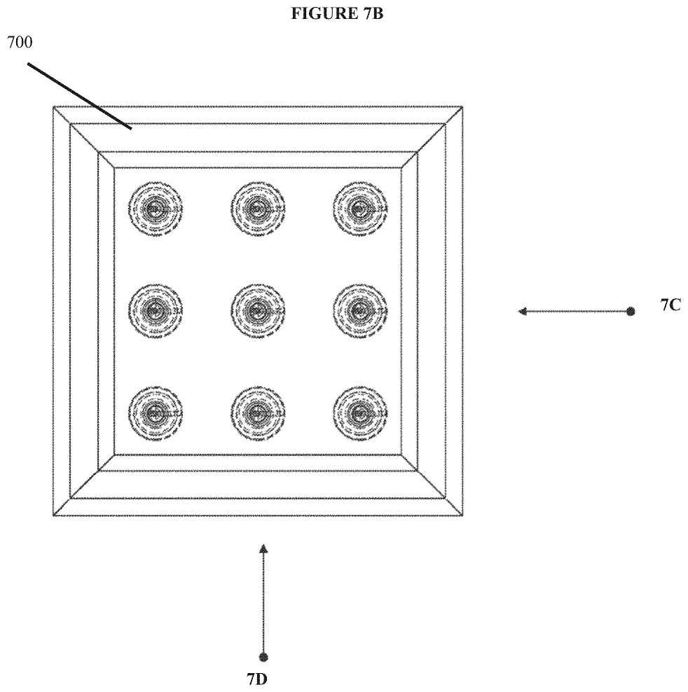

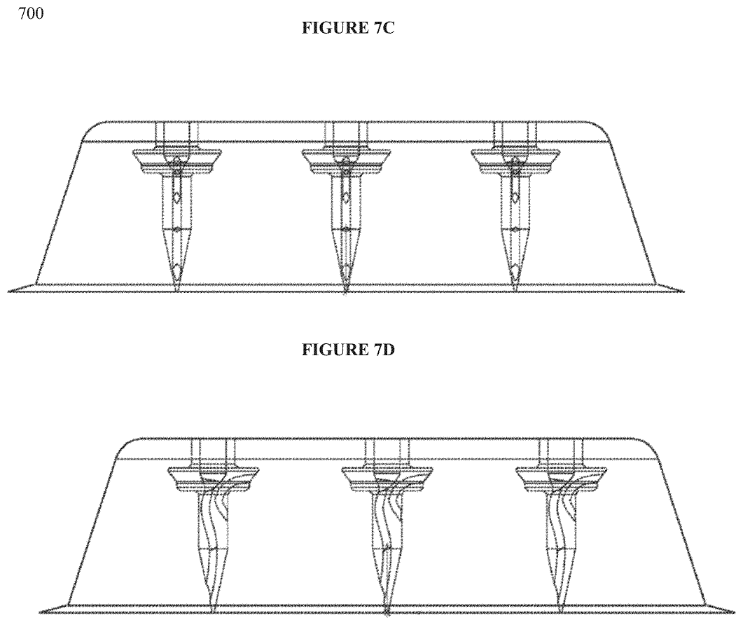

FIG. 7A shows a perspective schematic view of mold 700 for manufacturing a plurality of microneedles. As illustrated, in one embodiment, the mold comprises multiple cavities 701-709 arranged in a 3 by 3 array (for example but not limitation) for preparing microneedles according to this disclosure. The microneedles in this embodiment are similar in design to the microneedles shown in FIGS. 1 through 6. The microneedles manufactured in mold 700 are metallic. A first lumen provides fluid communication from a first portion of the base of the microneedle to a first locus proximate to the tip of the microneedle and a second lumen provides fluid communication from a second portion of the base of the microneedle to a second locus on the shaft of the microneedle. FIG. 7B shows a top schematic view of the microneedle mold. FIG. 7C shows a side schematic view of the microneedle mold, viewed along line 7C shown in FIG. 7B. FIG. 7D shows a side schematic view of the microneedle, viewed along line 7D shown in FIG. 7B.

FIG. 8 shows top and cross-section schematic views of a microneedle mold according to an embodiment of this disclosure, similar to the embodiment shown in FIG. 8A-8D. The upper image shows a top schematic view of the microneedle mold, wherein solid lines denote visible aspects of the microneedle mold and dashed lines denote invisible aspects of the microneedle, for example portions of the microneedle cavities on the interior of the microneedle mold. The bottom image shows a cross-section view of the microneedle mold wherein the structure of the microneedle mold has been cut away along line B-B shown in the upper image. The manufacturing process can be summarized as described below.

High precision 3D molds can be manufactured via two-photon lithography or two-photon polymerization. The fabrication method starts with manufacturing of the molds on a conductive substrate. We have shown that microneedles with 0.5 micron radius of curvature tips can be manufactured. Using the same order of magnitude feature sizes, the molds are made in the negative image of the desired needles by curing photoresist using two-photon lithography. The uncured photoresist is then stripped away by means of chemical treatment to leave cavities or voids in the mold. The substrate would then be left bare only at locations where the tips of the microneedles will be.

The substrate needs to have a conductive surface to enable electrodeposition of metal. Every part of the conductive surface, except for the bare parts inside the patterned molds will need to be masked before being submerged into the electrolyte for electrodeposition. The mold-substrate assembly would then be submerged into an electrolyte and electrochemical deposition of metal would occur, filling the cavities or voids inside the molds, taking the shape prescribed by the voids.

After submersion, electrodeposition is conducted by flowing current or applying voltage to the system, growing the needles, such as by tip-first, into the voids of the molds. The current density may be controlled by predicting the necessary current through mathematical models or keeping a constant voltage throughout the process.

Electrochemical deposition of metal into the mold can be accomplished using a rotating disk electrode. The bulk of the microneedle can be made by electrodeposition of a metal such as copper.

After the molds are filled with metal, the molds can be stripped away by means of heat treatment or chemical treatment. The metal needles may be released as a final step by electropolishing or etching the underlying layer of material. Because copper is not biocompatible, the microneedles are treated to put conformal layers of 1.5 microns of nickel and 100 nm of immersion gold to ensure biocompatibility. Conformal coating can be verified via energy-dispersive X-ray spectroscopy.

Hence, full metal needles will be manufactured in the shape of the voids inside the 3D lithographed molds, and this process in itself provides design freedom that is well suited and sometimes critical for a variety of applications.

In some embodiments, the microneedle(s) may be prepared in an array comprising a plurality of microneedles on a conductive substrate.

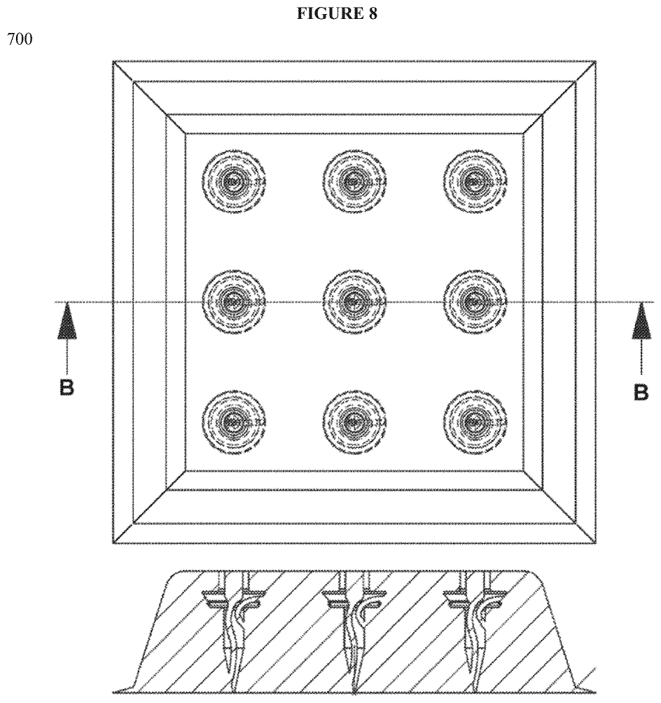

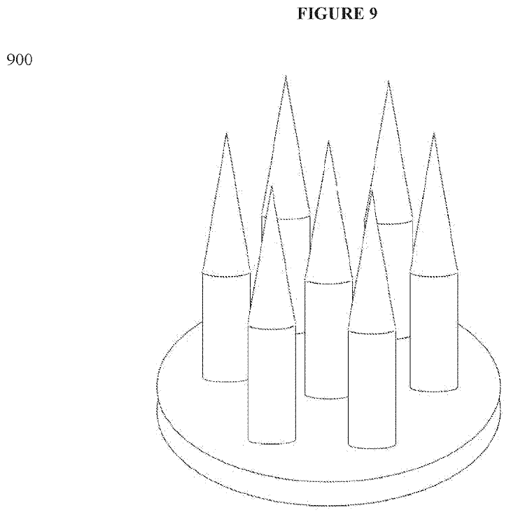

The microneedles of this disclosure may be used singly or as part of an array of a plurality microneedles 900 on a unitary base such as shown in FIG. 9. In the embodiment of FIG. 9, seven microneedles are shown but any number of microneedles may be used such as from 2 to 10 or more The unitary base may include a portion in fluid communication with the injection lumens of the plurality of the microneedles and a reservoir holding a therapeutic agent or replacement fluid, such as artificial perilymph for inner ear applications, depending on whether the microneedles are used for delivery of a therapeutic agent or for sampling a subject's bodily fluid. A second portion of the unitary base may be in fluid communication with the aspiration lumens of the plurality of the microneedles and a vacuum source and a receptacle for collecting fluids aspirated from the distal side of the membrane.

Various materials may be used to manufacture the microneedle. For example, in some embodiments, the microneedles are composed of only metallic material. For example, the microneedle may be fabricated from copper and may have a coating, such as but not limited to metals, ceramics, polymers, organics, etc. In one embodiment, the coating is gold. The coatings could consist of multiple layers and multiple materials and material classes.

### Example 1

In accordance with the subject matter described herein, multiple simultaneous perforations were made in round window membrane (RWM) of a guinea pig with an array of multiple needles. The use of such needle safely allows for greater diffusion of a therapeutic in a single setting without creating damaging tears between each perforation.

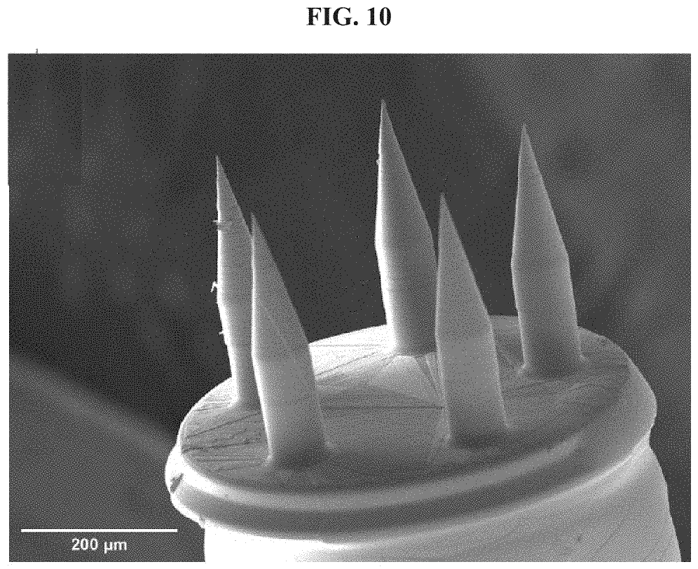

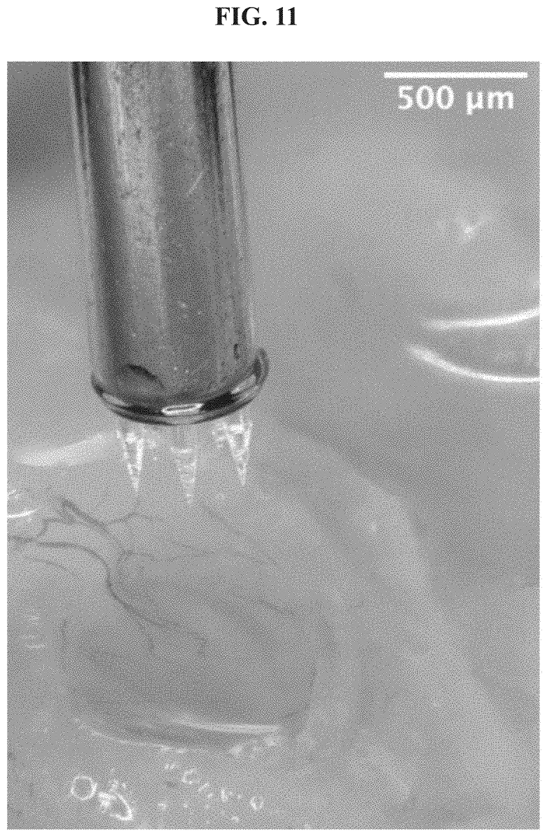

In the experiments, three variations of ultra-sharp polymer microneedle arrays were designed using Two-Photon Polymerization lithography (2PP) to be able to concurrently produce 3, 4, or 5 perforations in the guinea pig RWM (n=4 per group) in vitro. A five needle array is illustrated in FIG. 10, and illustrated approaching a guinea pig RWM in FIG. 11.

RWMs extracted from adolescent guinea pigs were perforated using mircroneedle arrays with three, four or five needles. RWMs were perforated with 100 μm diameter needles using a microindenter setup (n of 12). Perforation locations depended on the surgical anatomy of each RWM. The microindenter setup consists of a motorized linear translator to which the microneedle is secured (Zaber Technologies Inc., Vancouver, British Columbia, Canada) and a force transducer to measure the axial force exerted on the microneedle during perforation (Transducer Techniques, Temecula, Calif.). For stabilization the RWMs were bonded to plastic 3D-printed stages using dental cement.

Confocal Microscopy. Each RWM was analyzed using confocal microscopy postperforation to determine the effects of multiple simultaneous perforations on the membrane. Confocal image analysis was conducted using ImageJ-Fiji software. Perforation sizes were measured based on a maximum intensity projection image. Measurements consisted of perforation area, major axis, and minor axis.

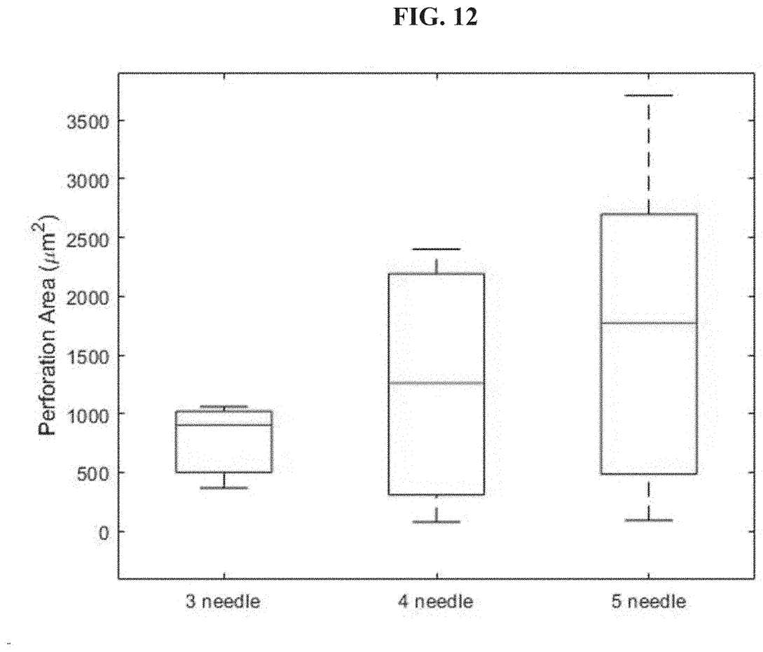

In performing the experiments, it was found that the microneedle arrays created multiple simultaneous perforations in the RWM without tearing between the perforations. The perforations are lens shaped, and the major axis is on average slightly smaller than the microneedle diameter. The microneedle array perforation area is shown in FIG. 12 for the 3-, 4-, and 5-microneedle arrays. As can be seen in FIG. 12, the total average perforation area increases with the number of needles in the array.

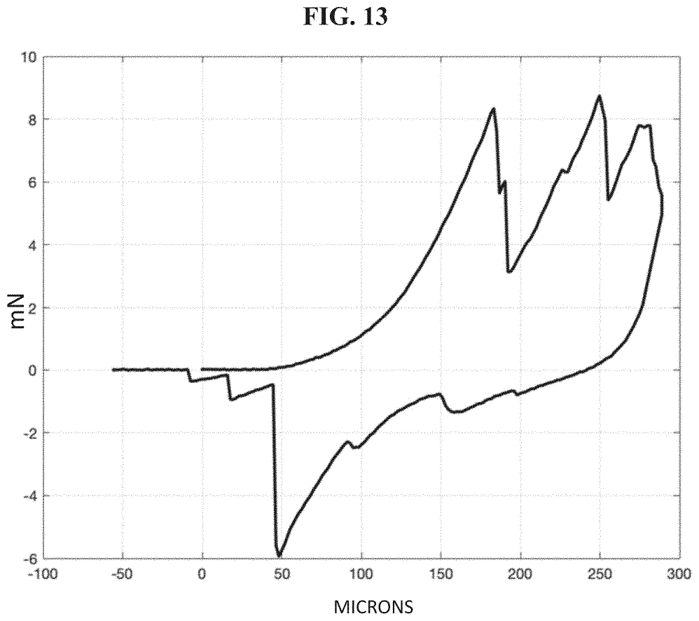

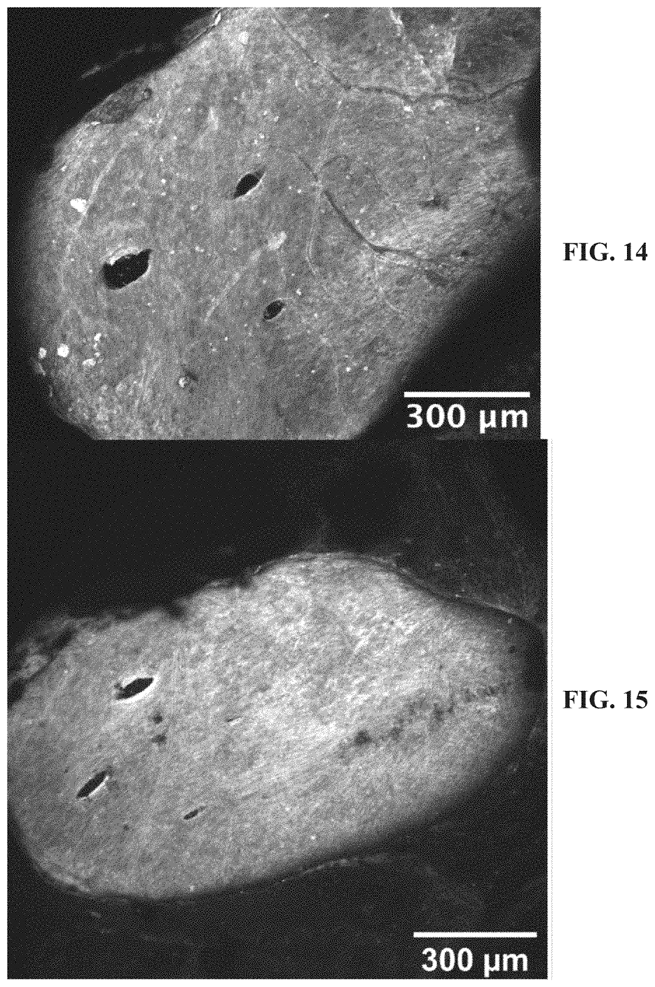

FIG. 13 illustrates the force (mN) vs. displacement (microns) of the 5 needle array. Positive force in the graph indicates a compressive force in the microneedle, i.e., when performing an indentation or perforation. Typically each peak positive force indicates a single perforation event, but there may be cases where multiple needles make simultaneous perforations. The peak forces are all similar, which demonstrates uniformity of the sequential perforation process. Negative force, i.e., the needle is in tension, occurs when the needle array is pulled out of the perforation. Friction between the RWM and the microneedle lead to the negative force. The area within the hysteresis loop quantifies the total energy dissipated (sum of creation of new RWM surface area and friction work). The fracture toughness of the RWM can be quantified from the hysteresis area. Minimization of the hysteresis area is an objective measure of needle design/quality. The sharper the microneedle, the lower the peak force. A smoother (and/or lubricated) needle shaft will result in a lower magnitude negative force upon pull-out. FIGS. 14-15 illustrate confocal images of the RWM showing perforations made by the 4-needle array.

3D-printed microneedle arrays can create multiple precise perforations in the RWM without tearing between perforations. The perforations were similar in size to those that have been shown to heal within 72 hours. Creating multiple perforations would allow for an increased total area of perforation and therefore allow for an increased delivery of therapeutic to the inner ear.

### Example 2

Glucocorticoids are the first-line treatment for sensorineural hearing loss, but little is known about the mechanism of their protective effect or the impact of route of administration. The recent development of hollow microneedles as described herein enables safe and reliable sampling of perilymph for proteomic analysis. Using these microneedles, the effect of intratympanic (IT) versus intraperitoneal (IP) dexamethasone administration on guinea pig perilymph proteome was investigated. Guinea pigs were treated with IT dexamethasone (n=6), IP dexamethasone (n=8), or untreated for control (n=8) 6 h prior to aspiration. The round window membrane (RWM) was accessed via a postauricular approach, and hollow microneedles were used to perforate the RWM and aspirate 1 μL of perilymph. Perilymph samples were analyzed by liquid chromatography-mass spectrometry-based label-free quantitative proteomics. Mass spectrometry raw data files have been deposited in an international public repository (MassIVE proteomics repository at https://massive.ucsd.edu/) under data set #MSV000086887. In the 22 samples of perilymph analyzed, 632 proteins were detected, including the inner ear protein cochlin, a perilymph marker. Of these, 14 proteins were modulated by IP, and three proteins were modulated by IT dexamethasone. In both IP and IT dexamethasone groups, VGF nerve growth factor inducible was significantly upregulated compared to control. The remaining adjusted proteins modulate neurons, inflammation, or protein synthesis. Proteome analysis facilitated by the use of hollow microneedles shows that route of dexamethasone administration impacts changes seen in perilymph proteome. Compared to IT administration, the IP route was associated with greater changes in protein expression, including proteins involved in neuroprotection, inflammatory pathway, and protein synthesis. Our findings show that microneedles can mediate safe and effective intracochlear sampling and hold promise for inner ear diagnostics.

Glucocorticoids, given systemically via oral route or locally by intratympanic (IT) injection, are the first-line treatment choice for sudden sensorineural hearing loss, yet little is known about the effects exerted in the inner ear and the mechanism of hearing loss treatment. Immunosuppressive effects via the glucocorticoid receptor have often been credited for their positive effects on hearing loss, but other cellular functions may also play a role. Furthermore, differences in effect between systemic and local administration of glucocorticoids are not fully elucidated. IT administration has been shown to be as effective as systemic administration in treating hearing loss. Local administration results in greater perilymph concentrations and favors the cochlear base, whereas systemic administration favors the apex. In mice, IT glucocorticoids affect thousands more inner ear genes compared to systemically delivered glucocorticoids on studies of mRNA expression in cochlear tissues.

Prior research on the effects of inner ear glucocorticoid administration has focused on their alteration of gene expression. Systemically administered glucocorticoids were found to modulate immune and inflammatory pathways, including genes that were associated with cytokine-cytokine receptor interaction and cell adhesion molecules in the immune system. Immune-related genes Ccl12 and Glycam1 were upregulated in noise-exposed cochlea but downregulated by systemic dexamethasone administration. Anti-inflammatory effects through binding of the glucocorticoid receptor are among possible mechanisms of action; however, effects through binding of the mineralocorticoid receptor as well as effects unrelated to inflammation may also be significant. Glucocorticoids have a strong binding affinity for the mineralocorticoid receptor and may exert effects on ion and water homeostasis within the inner ear. Another study found that ion homeostasis genes were upregulated after IT administration of glucocorticoids compared to control, while inflammatory cytokines were upregulated in both control and treatment groups, suggesting that effects through the mineralocorticoid receptor maybe predominant.

At the proteomic level, heat shock protein 70 and myelin protein zero in mice cochleae were found to be modulated by the administration of dexamethasone. However, this proteomic study required dissecting the whole cochlea from animals for an adequate tissue sample.

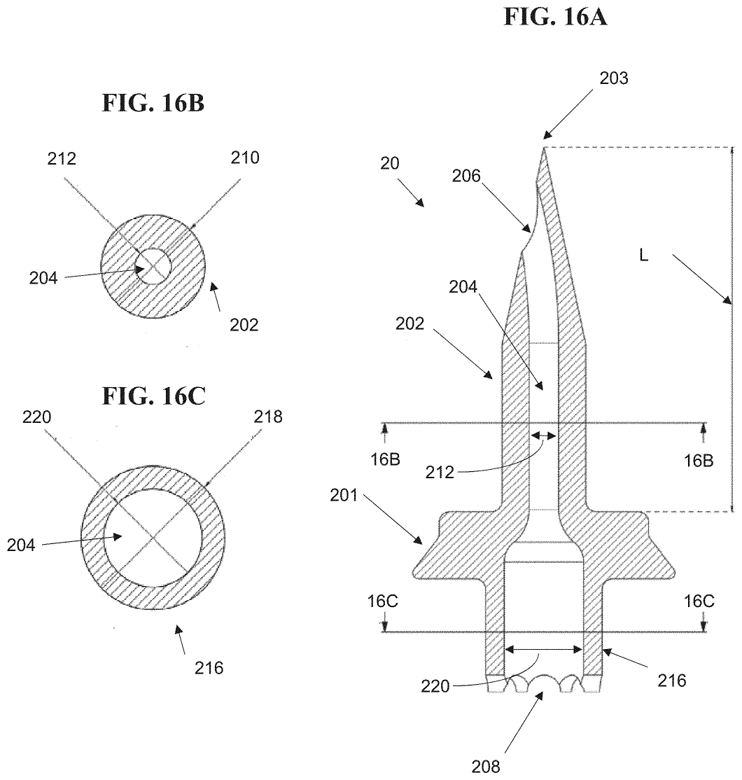

Our laboratory has developed novel microneedle technology for inner ear diagnosis and therapy based on the mechanical properties of the round window membrane (RWM). An additive manufacturing technique, two-photon polymerization lithography (2PP), was used to direct-write the microneedles via three-dimensional (3D) printing. Solid microneedles fabricated using this technique introduced microperforations in vivo that healed within 1 week without functional consequences. Hollow microneedles (FIG. 16) were then successfully used for atraumatic aspiration of perilymph across the RWM at a quality and volume adequate for proteomic analysis without causing lasting anatomic and functional dysfunction. These hollow microneedles could be used to sample perilymph of untreated guinea pigs and glucocorticoid-treated guinea pigs for comparison using liquid chromatography/mass spectrometry. In guinea pigs, perilymph aspirates may contain cerebrospinal fluid, but changes in protein expression should primarily reflect changes in the perilymph proteome. The purpose of this study is to determine the effects of IT and intraperitoneal (IP) dexamethasone on the perilymph proteome by utilizing 3D printed hollow microneedles to aspirate perilymph from dexamethasone-treated and untreated guinea pigs for proteomic analyses.

MATERIALS AND METHODS. With the exception of dexamethasone treatment, the majority of these methods have been previously published and are summarized here. Experiments were completed over the course of 4 months, alternating between control, IP dexamethasone (IP Dex)-treated, and IT dexamethasone (IT Dex)-treated guinea pigs to avoid freeze time bias of results. Animals were treated and sampled independently, and perilymph samples were stored at −80° C. and processed for proteomics analysis upon collection of all samples.

Microneedle Design and Fabrication. SolidWorks software (Dassault Systems SolidWorks Corporation, Concord, N.H.) was used for the computer-aided design of microneedles. Stereolithography files were generated and parsed using the Describe software (Nanoscribe GmbH, Karlsruhe, Germany), with a slicing distance of 1 μm and laser intensity of 80%. Microneedles were fabricated using 2PP by Photonic Professional GT system (Nanoscribe GmbH) using photoresist IP-S(Nanoscribe GmbH).

The microneedles 200 used in the experiment are illustrated in FIGS. 16A-C, and are substantially identical to the needle 10 illustrated in FIG. 3 above. The shaft 202 has a length L of 435 μm extending from the base 201 to the tip 203. The first section of the shaft 202, as illustrated in FIG. 16B is defined by a cylindrical, hollow body having a substantially uniform exterior diameter 210 of 100 μm and an interior lumen 204 having a diameter 212 of 35 μm. The second section, illustrated in FIG. 16A is defined by a conical, shaped body defined by a taper along a portion of its length terminating at a sharp or pointed tip 203. In some embodiments, the tip is closed, i.e., does not have a port or opening. In some embodiments depending on the application, the microneedle may a maximum tip diameter of about 10-20 microns. In embodiments having a port, lumen 204 extends from port 206 in the second section of the shaft 202 and curves to extend through the central axis of the needle 20 through to or through at least a portion of base 201, and has a second opening 208 disposed in base 201.

Base 201 is configured to mount to a commercially available standard blunt syringe. In this regard, second opening of lumen is in fluid communication with syringe and provides a channel for passage of therapeutic agent from syringe through lumen 204 to port 206 and exterior of microneedle 20 or alternatively, to drain/sample fluid through the port 206 and into lumen 204, and subsequently into the syringe structure.

As illustrated in FIG. 16C, the lumen 204 widens at the base 210 of the needle 20 to decrease fluidic resistance. The base 210 includes a proximal base portion 216 designed to mate with a syringe, such as a 30 G stainless steel syringe needle. Proximal base portion 216 has a substantially uniform exterior diameter 218 of 139 μm and the interior lumen 204 in this portion of the needle 20 having a diameter 220 of 95 μm.

Dexamethasone Treatment of Animals. All animal procedures described in this study were reviewed and approved by the Columbia University Institutional Animal Care and Use Committee. Twenty-two juvenile guinea pigs of either sex weighing between 150 and 300 g were obtained from a commercial vendor (Charles River Laboratories, Inc., Wilmington, Mass.).

Eight animals were assigned to the control group and were not given any treatment prior to aspiration. Eight animals were assigned to the IP Dex group, and six animals were assigned to the IT Dex group. The audiometric and healing data of four guinea pigs (two control, two IP Dex) were not available for analysis because of equipment malfunction unrelated to aspiration; thus, two additional guinea pigs were assigned to the control and IP Dex groups. Dexamethasone sodium phosphate (10 mg/mL, MWI Veterinary Supply, Boise, Id.) was administered to IP Dex guinea pigs 6 h prior to the time of aspiration, by IP injection at a dose of 10 mg/kg. It was demonstrated that both IT and IP dexamethasone administration resulted in dexamethasone labeling of the cochlea at 6 h, and the time point of 6 h has previously been chosen for dexamethasone administration experiments. For IT Dex guinea pigs, all IT injections were applied to the right ear 6 h prior to the time of aspiration. Guinea pigs were anesthetized with isoflurane gas (3.0% for induction and 1.0-3.0% for maintenance). A 27 gauge needle was used to first create a ventilation hole in the anterior-superior quadrant of the tympanic membrane. It was then used to inject 50 μL of the 10 mg/mL dexamethasone sodium phosphate solution (equating to 0.5 mg) through the posterior-superior quadrant of the tympanic membrane into the middle ear space. The guinea pig remained under isoflurane anesthesia in a left lateral decubitus position for 30 min. The guinea pig was then allowed to recover in its cage until the aspiration procedure.

Aspiration Procedure. All guinea pigs underwent the aspiration procedure detailed below. For the IP Dex and IT Dex animals, aspiration was performed 6h after dexamethasone treatment. All procedures were performed on the right ear. Guinea pigs were anesthetized with isoflurane gas (3.0% for induction and 1.0-3.0% for maintenance). Lidocaine was injected subcutaneously for local anesthesia. Buprenorphine sustained release (0.1 mg/kg) and meloxicam (0.5 mg/kg) were both administered subcutaneously prior to surgery for postoperative analgesia. To reduce head motion from breath cycles, head fixation was achieved using a modular 3D-printed head holder with two pointed screws placed anterior to the external auditory meatus and posterior to the orbit without piercing the skin.

A scalpel was used to create a 1 cm postauricular incision. Blunt dissection was used to expose the bulla. A 1 mm drill tip fixed onto a Stryker S2 πDrive drill (Stryker, Kalamazoo, Mich.) was used to create a small opening into the middle ear space. Additional bone was removed using forceps to enlarge the opening to 2-3 mm in diameter for optimal visualization of the RWM. A hollow microneedle, mounted onto the tip of a 2 in. long, 30 gauge, blunt small hub removable needle (Hamilton Company, Reno, Nev.), was secured onto a 10 μL Gastight Hamilton syringe (Model 1701 RN, Hamilton Company, Reno, Nev.). The syringe was mounted onto a UMP3 UltraMicroPump (World Precise Instruments, Sarasota, Fla.), and the pump was fixed to a micromanipulator. Using the micromanipulator, the mounted hollow microneedle was slowly advanced toward the RWM and the perforation was confirmed by visualization.

Using the UltraMicroPump, 1 μL of perilymph was aspirated across theRWMover 45 s. The sample was ejected into a 0.5 mL LoBind Microcentrifuge tube (Eppendorf, Hamburg, Germany) containing 2 μL of 1% protease cocktail inhibitor solution (P8340, Sigma-Aldrich, St. Louis, Mo.) in liquid chromatography/mass spectrometry grade water (Optima, ThermoFisher Scientific, Fair Lawn, N.J.) and stored at −80° C. Seventy-two hours after aspiration, animals were sacrificed with pentobarbital overdose.

Audiometric Testing. Audiometric testing was performed under anesthesia using methods previously described. Compound action potential (CAP) was used to evaluate the animal's hearing before RWM perforation and also immediately prior to euthanasia at 72 h after completion of the procedure. Distortion product otoacoustic emissions (DPOAE) were used to evaluate hearing before and after RWM perforation and immediately prior to animal sacrifice at 72 h after the procedure.

CAP measures the activity of the auditory nerve by recording the synchronous firing of the sum of each individual unit action potential near the RWM after sound stimulation. Tone pips were played into the ear, and the neural responses were measured by a silver electrode placed at the base of the cochlea. CAP responses were measured for 18 frequencies ranging from 0.5 to 40 kHz. Stimulus intensity was steadily increased in 5 dB increments to determine a hearing threshold. The lowest stimulus level that induced a recognizable response curve was used to determine the threshold. To minimize bias, experimenters were blinded to previous threshold measurements. CAP threshold shifts were considered significantly different than zero at p<0.05 using two tailed paired t-tests.

DPOAE are responses produced by the cochlea upon simultaneous stimulation by two pure tone frequencies. They are used to measure the health of outer hair cells and to evaluate potential levels of hearing loss. To perform DPOAE measurements, an ear tube containing a speaker and a low-noise Sokolich ultrasonic probe microphone was placed into the ear canal. The speaker played sound stimuli at sound pressure levels of 70 dB SPL with a fixed frequency ratio off 2/f1=1.2 at 1 kHz increments between 1 and 32 kHz. The microphone detected resulting distortion products from the ear. A DPOAE at 2f1-f2 that is 3 dB above the noise floor level was identified as a positive response.

Proteomics Analysis. All perilymph samples (1 μL) were stored at −80° C. and then processed as described previously. Briefly, samples were purified with a methanol chloroform protein precipitation as previously described. Proteins were resuspended in 8 M urea, 3 mM dithiothreitol (DTT), 100 mM ammonium bicarbonate in liquid chromatography/mass spectrometry grade water, reduced with dithiothreitol, and alkylated with iodoacetamide. For proteolytic digestion, samples were diluted 5-fold in 100 mM ammonium bicarbonate and then digested using sequencing grade trypsin (Promega V511) at a protease/protein ratio of 1:50 at 37° C. for 16 h as described previously. Samples were then desalted with Nest Group C18 Macrospin columns (Southborough, Mass.). Resulting peptides were analyzed by liquid chromatography/mass spectrometry as described previously. Peptide concentration was evaluated by NanoDrop spectrophotometry (ThermoFisher Scientific) at 205 nm and LC/MS inject loading amounts were adjusted (normalized) based on amino acid concentration. Specifically, separations were performed with an Ultimate 3000 RSLCnano liquid chromatograph with a 75 μm IDX 50 cm Acclaim PepMap C18 column (Thermo Scientific P/N 164942) coupled to a Q Exactive HF mass spectrometer (Thermo Scientific) in positive ion mode using data-dependent acquisition. Acquisition settings included top 15 precursors, resolution 120 000 for MS scan, 15 000 for MS/MS scan, dynamic exclusion for 20 s, and maximum injection time of 30 ms for MS and 100 ms for MS/MS. For fragmentation, NCE was set at 28.0. The Nanospray Flex Ion Source was operated at 2.2 kV with a heated capillary set at 250° C. and the S-Lens RF level at 55.0%.

Raw data files were searched with MaxQuant Version 1.6.10.43 with Andromeda search engine with FDR 0.01 for PSM and Protein. Fixed modification was carboxyamidomethylation of cysteine and variable modifications were oxidation (M) and acetyl (protein N-term). The database was UniProt release 2019_10, published Nov. 13, 2019 with 25 731 sequences 14 311 265 residues of reviewed and unreviewed Cavia porcellus sequences and isoforms. C. porcellus sequences were from reference proteome #up000005447 and included porcine trypsin, human keratins, and lab contaminants. Search parameters included MS tolerance of 20 ppm and MS/MS tolerance of 20 ppm. Statistical post processing was with Perseus Version 1.6.10.50. Label-free quantitation (LFQ) in MaxQuant was used. Normalization of data across runs is performed by the LFQ algorithm. Statistical analysis of LFQ data was performed in Perseus with analysis of variance (ANOVA) and Tukey's honestly significant difference post-hoc test. All mass spectrometry raw data files generated in this work have been deposited in an international public repository (MassIVE proteomics repository at https://massive.ucsd.edu/) under data set #MSV000086887.

Characterizing the Guinea Pig Perilymph Proteome in Treated and Untreated Guinea Pigs. Perilymph from 22 guinea pigs was included for proteomic analysis. Identifications were returned for 632 proteins with a 1% false discovery rate for both peptide sequence matches and protein identifications. Of the 632 proteins, 414 proteins represented by a single peptide or with insufficient data (<3 data points per treatment group) were deleted from the final analysis. An additional 43 proteins were contaminants or added proteins. One hundred seventy-five proteins represented by two or more peptides were included in the analysis.

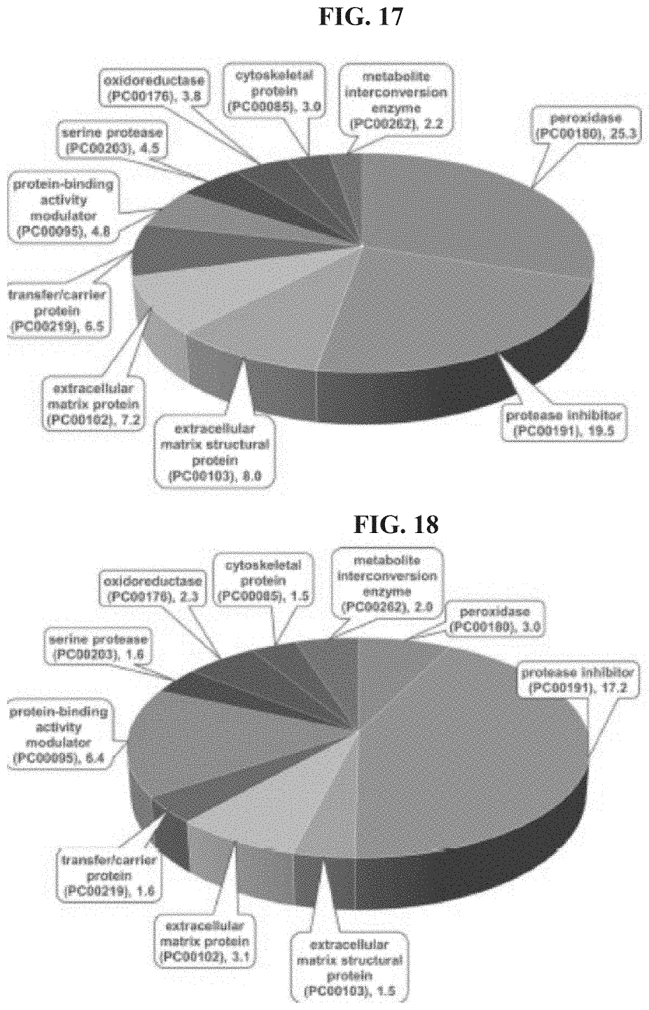

Data were analyzed with the PANTHER ontology tool (http://www.pantherdb.org) using gene names searched against a Mus musculus background. FIGS. 17-18 illustrate the composition of guinea pig perilymph protein by function categories. The number of proteins per functional category and the distribution of these functional categories are displayed, accounting for protein abundances. 175 gene names were searched against the mouse gene list in PANTHER to determine the distribution of proteins across functional classes. One hundred forty-six proteins were mapped onto a functional category in PANTHER. Among these 146 proteins, the most common functional categories are protease inhibitors, protein-binding activity modulators, extracellular matrix proteins, and peroxidases. FIG. 17 illustrates the protein class fold-enrichment. FIG. 18 illustrates the protein class −log 10 FDR enrichment.

At the individual level, preproalbumin, globin A1, hemoglobin subunit a, transthyretin, α-2-HS-glycoprotein, and Serine proteinase inhibitor A3K were the most abundant proteins in all groups. The well-known inner-ear protein cochlin (A0A2C9F1F1_CAVPO) was identified based on 10 unique peptides, with 26% sequence coverage, was also a high abundance protein.

Modulated Proteins of Each Treatment Group Overall, 15 proteins were modulated by either IP Dex or IT Dex compared to control. Compared to control, 14 proteins were modulated in the IP Dex group, 8 were upregulated, and 6 were downregulated (Table 2).

TABLE 2

| | | | abundance | p-value (IP |
|---|---|---|---|---|
| | | | ratio (IP | Dex versus |
| Uniprot ID | protein | gene name | Dex/control) | control) |
| H0VZA9_CAVPO | VGF nerve growth factor inducible | VGF | 3.20 | 1.6 × 10⁻³ |
| H0V8E0_CAVPO | amyloid β precursor like protein 1 | APLP1 | 2.44 | 6.9 × 10⁻³ |
| A0A286XP66_ | tyrosine 3- | YWHAG | 0.42 | 8.1 × 10⁻³ |
| CAVPO | monooxygenase/tryptophan 5- | | | |
| | monooxygenase activation protein γ | | | |
| H0W9I8_CAVPO | elongation factor 1-α | | 0.38 | 0.01 |
| H0W3T8_CAVPO | fructose-bisphosphate aldolase | ALDOC | 0.26 | 0.02 |
| H0UW89_CAVPO | SPARC like 1 | SPARCL1 | 3.82 | 0.03 |
| H0VLM3_CAVPO | heat shock protein family A | Hspa5 | 0.67 | 0.03 |
| | (Hsp80) member 5 | | | |
| H0V7P8_CAVPO | annexin | ANXA2 | 0.59 | 0.04 |
| H0UY41_CAVPO | complement C5 | C5 | 3.56 | 0.04 |
| H0V7V7_CAVPO | Dickkopf WNT signaling pathway | DKK3 | 1.97 | 0.04 |
| | inhibitor 3 | | | |
| H0VN20_CAVPO | enolase 3 | ENO3 | 0.09 | 0.04 |
| H0V7Q6_CAVPO | neuronal cell adhesion molecule | NRCAM | 3.34 | 0.04 |
| H0V077_CAVPO | SMB domain-containing protein | Vtn | 1.57 | 0.05 |
| H0UV55_CAVPO | neural EGFL-like 2 | NELL2 | 1.78 | 0.05 |

Only three proteins were modulated in the IT Dex group; one was upregulated and two were downregulated (Table 3).

TABLE 3

| | | | abundance | p-value (IT |
|---|---|---|---|---|
| | | | ratio (IT | Dex versus |
| Uniprot ID | protein | gene name | Dex/control) | control) |
| H0VZA9_CAVPO | VGF nerve growth factor | VGF | 2.23 | 7.0 × 10 − 3 |
| | inducible | | | |
| A0A286XP66_ | tyrosine 3- | YWHAG | 0.41 | 0.03 |
| CAVPO | monooxygenase/tryptophan 5- | | | |
| | monooxygenase activation | | | |
| | protein γ | | | |
| H0W263_CAVPO | Insulin-like growth factor | IGPBP2 | 0.60 | 0.03 |
| | binding protein 2 | | | |

VGF nerve growth factor inducible (VGF) was significantly upregulated compared to control in both IP (abundance ratio 3.20, p=0.0016) and IT (abundance ratio 2.23, p=0.007) Dex groups. Tyrosine 3-monooxygenase/tryptophan 5-monooxygenase activation protein γ was significantly downregulated in both treatment groups (IP: abundance ratio 0.42, p=0.0081; IT: abundance ratio 0.41, p=0.03).

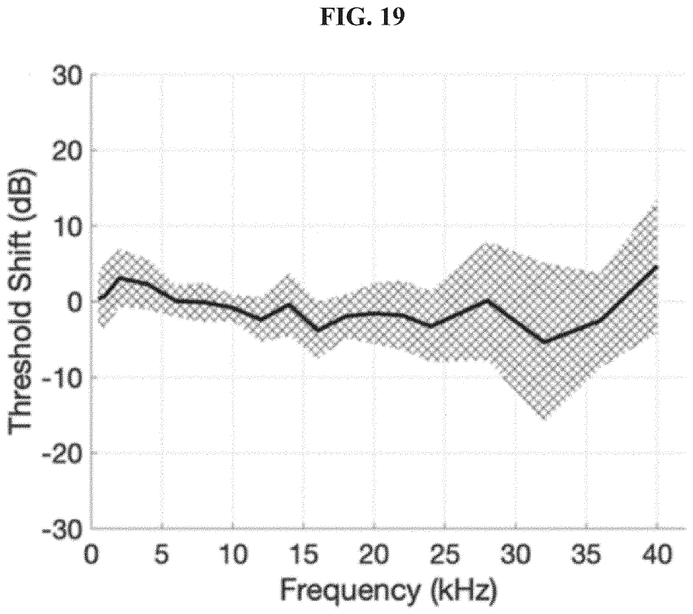

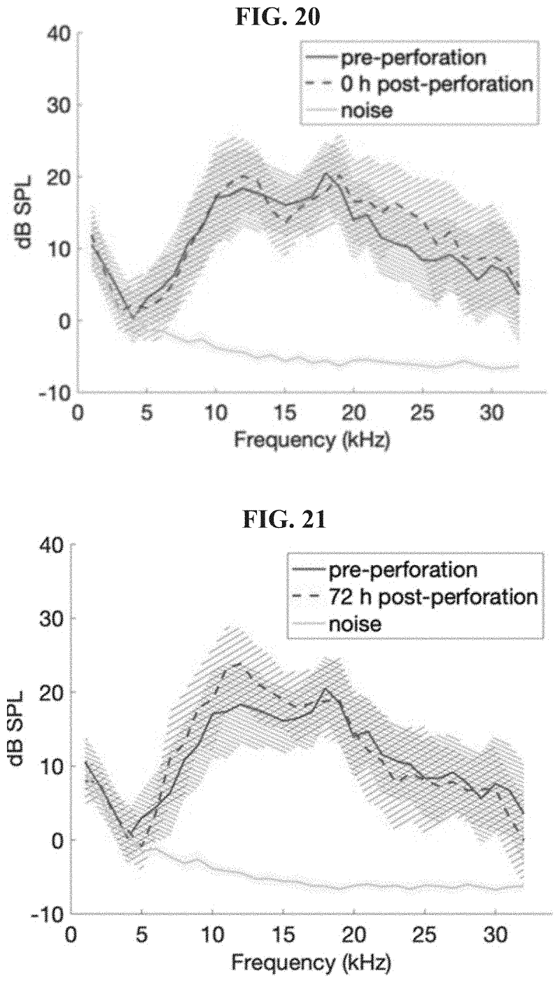

Audiometric Testing and RWM Healing. Aspiration resulted in no permanent damage to hearing at 72 h and all perforations healed completely within 72 h. Of the 18 guinea pigs (n=6 per treatment group) that underwent all audiometric measurements successfully, there was no permanent damage to hearing at 72 h after perforation as assessed by Compound action potential (CAP) and distortion product otoacoustic emissions (DPOAE), and all perforations healed completely within 72 h. Results were similar between control, IP Dex, and IT Dex groups. FIG. 19 illustrates Mean CAP threshold shifts at 72 h compared to baseline, for all tested frequencies. The shaded area is bounded by the upper and lower limits of the 95% confidence interval. There was no significant shift in CAP measured at 72 h after perforation, for all 18 frequencies (n=18). FIGS. 20-21 illustrate DPOAE at the frequency 2f1-f2 in response to a 70 dB stimulus averaged over all survival experiments at 0 h after perforation (A, n=18) and 72 h after perforation (B, n=18). Solid gray lines show the average noise for the experiments; solid blue lines show the mean measured baseline DPOAE signal, and dotted red lines show the mean DPOAE signal at 0 h (FIG. 20) or 72 h (FIG. 21) postperforation. Shaded areas are bounded by the upper and lower limits of the 95% confidence interval for the average noise and mean DPOAE signals. All DPOAE signals remained out of noise.

In this study, using novel hollow microneedle technology, we show that the route of dexamethasone administration greatly impacts changes seen in perilymph proteome. The hollow surgical microneedles facilitated the aspiration of 1 μL of perilymph from the scala tympani in a series of survival experiments on guinea pigs; all RWMs healed within 72 h and aspiration did not cause lasting physiological dysfunction, further confirming previously published results in untreated guinea pigs. Among these 175 proteins represented by two or more peptides and detected in at least three samples per treatment group, 14 proteins were modulated by systemic dexamethasone and 3 proteins were modulated by IT dexamethasone. In both treatment groups, VGF was found to be upregulated and tyrosine 3-monooxygenase/tryptophan 5-monooxygenase activation protein γ (also known as 14-3-3 γ) was found to be downregulated.

The VGF polypeptide is the precursor of several biologically active peptides with expression restricted to a subset of neurons in the central and peripheral nervous system and specific populations of endocrine cells. VGF and its peptides are found in large dense core vesicles and released from neuronal and endocrine cells through the secretory pathway. VGF peptides include NERP-1 and NERP-2, NAPP129, TPGH, TLQP-21, TLQP-62, HHPD-41, AQEE-11, AQEE-30, and LQEQ-10 and have been shown to regulate neuronal activities such as synaptic plasticity, neurogenesis, and neurite growth. VGF also exhibits neuroendocrine effects through two of its peptides, NERP-1 and NERP-2, which modulate antidiuretic hormone release and may be important for fluid balance. VGF expression levels are very low under normal physiological conditions but are rapidly upregulated in various situations, such as nerve injury.

VGF is induced by neurotrophic factors, including brainderived neurotrophic factor (BDNF) and neurotrophin-3 (NT3). BDNF and NT3 have both been shown to exert otoprotective effects through preservation of spiral ganglion neuron and regeneration of cochlear synapses. It has also been suggested that VGF may have roles in neuroprotection. In an in vitro model of amyotrophic lateral sclerosis, VGF was shown to be a mediator of the protective effects of the free radical scavenger SUN N8075 on ER stress-induced cell death. In another study, VGF was shown to be induced by optic nerve crush. The VGF peptide, AQEE-30, suppressed the loss of retinal ganglion cells in mice with optic nerve crush and promoted outgrowth of neurites of rat- and human-induced pluripotent stem cells-derived retinal ganglion cells in vitro. In our study, we found that dexamethasone, a glucocorticoid commonly used to treat sudden sensorineural hearing loss, when systemically or intratympanically administered, induced protein expression of VGF. VGF may be a mechanism for the protective effects of dexamethasone, but further studies involving animal models for hearing loss, including VGF knock-outs and overproducers, are needed.

Interestingly, 14-3-3 γ was downregulated after both IP and IT dexamethasone administration. Similarly, in mice, 14-3-3 has been shown to increase in the meninges and CSF of mice with eosinophilic meningitis after infection, and was downregulated upon dexamethasone administration. It has also been found that 14-3-3 γ upregulates glucocorticoid receptor in the liver. Our findings show that treatment with dexamethasone, a glucocorticoid, 14-3-3 levels in the perilymph are downregulated. Taken together, these results suggest a negative feedback loop between the 14-3-3 γ and glucocorticoids whereby 14-3-3 upregulates the glucocorticoid receptor and is, in turn, downregulated when glucocorticoid levels are high. However, it is unknown if 14-3-3 γ also upregulates the glucocorticoid receptor in the inner ear. Further studies of 14-3-3 γ in the inner ear are needed to elucidate its possible role as a mediator of the protective effect of glucocorticoids on hearing.

The remaining proteins modulated by either systemic dexamethasone or intratympanic dexamethasone can be broadly divided into three general categories. Six of these proteins, all upregulated following IP Dex, have roles in neurogenesis, neural development, or neuroprotection: APLP1, NRCAM, NELL2, DKK3, VTN, and SPARCL1. Amyloid β precursor like protein 1 (APLP1), part of the amyloid precursor protein family essential to central nervous system development and exclusively expressed in neurons, has roles in proper synapse formation and maintenance. Neuronal cell adhesion molecule (NRCAM) is expressed by sensory cells in the cochlea and is necessary for proper cochlear innervation during development. Neural EGFL-like 2 (NELL2) has roles in neural cell growth and differentiation. Dickkopf WNT signaling pathway inhibitor 3 (DKK3) is anti-inflammatory and neuroprotective in intracranial hemorrhage. Vitronectin (VTN) supports cell attachment and promotes neurite extension and has been shown to be upregulated in optic nerve injury. SPARCL1 regulates cell migration, proliferation, and differentiation and is upregulated in brain injury.

Heat shock protein, a protein involved in the inflammatory process, was downregulated in guinea pigs treated with systemic dexamethasone. Previously, consistent with our finding, heat shock protein family A (Hsp80) member 5 (Hspa5) was shown to be downregulated by dexamethasone in the mouse cochlea of a mouse model of hearing loss. Annexin A2 (ANXA2) was also downregulated in guinea pigs treated by IP Dex in the present study. ANXA2 is proinflammatory protein that activates inflammatory cytokines, in contrast to its counterpart Annexin A1, which has anti-inflammatory roles. In addition, Complement C5, the fifth component of complement with an important role in inflammation and cell killing processes, was upregulated by systemic dexamethasone in this study.

Finally, proteins involved in protein synthesis (Elongation factor 1-α), glucose metabolism (fructose-bisphosphate aldolase and enolase 3), and adipogenesis (Insulin-like growth factor binding protein 2) were all downregulated by dexamethasone in this study.

In comparing the effects of systemically delivered and IT administered dexamethasone, 14 proteins were modulated by IP Dex, while only three proteins were modulated by IT Dex. This suggests that IT Dex has narrower effects on the perilymph while IP Dex imparts broader results due to its systemic delivery route. There are few studies for comparison in this largely unexplored area; additionally, prior studies used different methodologies for assessing changes (e.g., inner ear tissues rather than perilymph), thus making direct comparison difficult. For example, Trune et al. showed that intratympanic dexamethasone administration changed the expression of many more inner ear genes than systemic dexamethasone injection based on mRNA expression in tissues, protein expression was not studied. Similar to our study, they also used a 6 h time point following dexamethasone administration; however, while they used whole cochlear tissue for analysis, we utilized perilymph for proteomic analysis. The authors pointed out that theirs was the first to examine the full inner ear genome impact of steroids, and that 75% of the genes affected, while statistically significant, were upregulated less than 2-fold or downregulated by less than 50%. The authors suggest that the actual change in expression was often minor and perhaps even unimportant in the overall function of the inner ear. No gene list was included in that paper so a detailed comparison to this work is not possible.

IT delivery of dexamethasone is limited by the inconsistent diffusion of drug across the RWM and inadvertent clearance of medication via the eustachian tube. Given these limitations, it is unclear whether the results of the current study suggest robustness of systemic delivery over local delivery or the vagaries of IT delivery. The rate of diffusion of medication delivered by IT injection may be improved by introducing perforations into the RWM using solid microneedles. Alternatively, direct intracochlear injection of medication may be considered to reduce the variability associated with RWM diffusion and eustachian tube leakage.

Furthermore, changes to protein expression may be time dependent; thus, proteomic analyses of perilymph conducted over time may yield valuable information and overcome the limitation of single time point study. In principle, our sampling method permits multiple perilymph aspirations from the same animal over time as perforations heal and cause no lasting damage to hearing. Future studies should focus on direct delivery across RWM and repeated sampling of perilymph at various time points to determine the effect of steroids over time.

In this study, dexamethasone was found to upregulate proteins involved in neural development, neuronal regulation, and neuroprotection; modulate proteins involved in inflammatory processes; and downregulate proteins involved in protein synthesis, glucose metabolism, and adipogenesis. VGF was upregulated in the perilymph of guinea pigs treated with IT and systemic dexamethasone and is possibly a mechanism of the protective effect of glucocorticoids on hearing loss. Hollow microneedles facilitated the aspiration of perilymph across the RWM at microliter volumes without causing permanent anatomic or physiologic dysfunction and enabled proteomic analysis. Future studies may investigate the effects of direct intracochlear injection of dexamethasone repeated over multiple time points.

Although the microneedle is described for the use the thin membrane of the inner ear, this application should not be construed as limiting. The microneedles described and embodied herein may be used for other applications. Further, the microneedle may be modified to suit a plethora of needs, for sampling or delivering across different physiological structures. One of ordinary skill in the art would also appreciate that the selection of materials to fabricate the microneedle may be modified to create more rigid structures to penetrate other membrane and anatomies.

While the disclosed subject matter is described herein in terms of certain non-limiting exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of non-limiting example embodiments of the disclosed subject matter has been presented for purposes of illustration and description. It is not intended to be exhaustive or to limit the disclosed subject matter to those embodiments disclosed herein

It will be apparent to those skilled in the art that various modifications and variations can be made in the method and system of the disclosed subject matter without departing from the spirit or scope of the disclosed subject matter. While the disclosures herein are described for accessing the cochlea, the issue of localized drug delivery across anatomic barriers to enclosed spaces extends beyond the field of otolaryngology.

What is claimed is:

1. A microneedle comprising:
a base disposed at a proximal end of the microneedle body and a tip disposed at the distal end of the microneedle body, a shaft having a longitudinal body disposed between the base and the tip, wherein the base has a width greater than the width of the tip,
at least one lumen defining a channel providing fluid communication and passage disposed within at least the shaft of the microneedle body, and
an opening defined at the distal end of the lumen and at a loci of the shaft, wherein the opening is off-set from the central longitudinal axis of the tip.

2. The microneedle of claim 1, wherein the at least one lumen has a body extending through the base to the shaft of the microneedle.

3. The microneedle of claim 1, wherein the at least one lumen has a body that terminates proximate to the tip.

4. The microneedle of claim 1, wherein the base is configured to mount a medical instrument.

5. The microneedle of claim 4, wherein the medical instrument is a syringe.

6. The microneedle of claim 1 wherein the base has a width greater than the shaft.

7. The microneedle of claim 1, wherein the base, shaft and tip are integral to form microneedle body.

8. The microneedle of claim 1, wherein the tip includes a sharp, pointed apex.

9. The microneedle of claim 1, wherein the offset is about 25 microns.

10. The microneedle of claim 1, wherein the tip has a 500 nm radius of curvature.

11. The microneedle of claim 1, wherein the shaft is about 350 microns.

12. The microneedle of claim 1, wherein the at least one lumen is only one sole lumen.

13. The microneedle of claim 1, wherein the at least one lumen is two separate lumens.

14. The microneedle of claim 1, wherein the maximum tip diameter is about 10-20 microns.

15. The microneedle of claim 1, wherein the shaft has a tapered longitudinal, cylindrical body configured with a taper along a portion of its length.

16. A microneedle comprising
a base,
a shaft,
a sharp tip and
two or more lumens wherein a first lumen provides fluid communication and passage from a first portion of the base of the microneedle to a first locus proximate to the tip of the microneedle and a second lumen provides fluid communication and passage from a second portion of the base of the microneedle to a second locus on the shaft of the microneedle.

17. The microneedle of claim 16 wherein the first locus and the second locus are diametrically opposed on the microneedle.

18. The microneedle of claim 16 wherein the microneedle has a maximum tip diameter of about 10-20 microns.

19. The microneedle of claim 16 wherein the microneedle is configured with a taper along a portion of its length.

20. The microneedle of claim 19 wherein the taper comprises a gradual taper having a gradual decrease in diameter along the length of the microneedle.

21. The microneedle of claim 19 wherein the taper comprises a stepped taper with abrupt changes in diameter that serve as reinforcing ribs or ledges.

22. The microneedle of claim 16, wherein a distal portion comprises a narrow sharp tip, the proximal end comprises a wide base and a shaft between the tip and the base.

23. The microneedle of claim 22 wherein the base of the distal portion may comprise one or more projections or barbs that engage the distal side of the membrane after penetration through the membrane and is held in place thereby.

24. The microneedle of claim 16, wherein the base is of the microneedle is configured to physically engage a driver device capable of creating temporary perforations in an anatomic membrane.

25. The microneedle of claim 16, configured to penetrate an anatomic membrane and simultaneously inject a first liquid into the distal side of the membrane and withdraw a second liquid from the distal side of the membrane.

26. The microneedle of claim 25 wherein the anatomic membrane is a membrane of the inner ear.

27. The microneedle of claim 25 wherein one lumen is an aspirating lumen which is connected to a suction device, such as a vacuum source, to suction a fluid from the posterior side of the membrane.

28. The microneedle of claim 16, wherein one lumen is an injection lumen which is connected to an injection device, such as a syringe, to inject a fluid into the posterior side of the membrane.

29. A medical device comprising a microneedle of claim 16, wherein the base the microneedle is physically engaged with a driver device capable of creating temporary perforations in an anatomic membrane and simultaneously inject a first liquid into the posterior side of the membrane and withdraw a second liquid from the posterior side of the membrane.

30. The medical device of claim 29 wherein the membrane is the round window membrane of an inner ear.

31. The medical device of claim 29 wherein the microneedle and the driver comprise separate components that are engaged to each other to define a modular system.

32. The medical device of claim 29 further comprising an indicator to indicate when the membrane is fully penetrated by the microneedle.
